(19) United States
(12) Patent Application Publication
(10) Pub. No.: US 2020/0152300 A1
(43) Pub. Date: May 14, 2020

(54) METHOD AND COMPUTER PROGRAM FOR PROVIDING A HEALTHCARE MANAGEMENT PLATFORM

(71) Applicant: Alexandra Coren, Miami, FL (US)

(72) Inventor: Alexandra Coren, Miami, FL (US)

(21) Appl. No.: 16/740,059

(22) Filed: Jan. 10, 2020

Related U.S. Application Data
(63) Continuation of application No. 15/348,251, filed on Nov. 10, 2016, now abandoned.
(60) Provisional application No. 62/415,838, filed on Nov. 1, 2016.

(51) Int. Cl. G16H 10/60; G16H 40/20; G06F 21/62

(52) U.S. Cl. CPC G16H 10/60; G16H 40/20; Y02A 90/26; Y02A 90/22; G06F 21/6245

(57) ABSTRACT

The present invention is directed to a method for providing a healthcare management platform. Specifically, an application server may be implemented to provide a website or application for access to various user portals of the platform while providing the facility's management with analytical tools such as real-time patient satisfaction. For example, a patient may be given access to a private patient portal in order to at least submit performance surveys of employees of the facility. A patient may designate guests, who may access the patient portal as would the associated patient. An employee may be given access to a private employee portal in order to at least redeem virtual currency awarded automatically via positive survey results. An administrator may be given access to an administrator portal in order to at least view employee survey data.

### CLAIM OF PRIORITY

The present application is a continuation application of previously filed, now pending application having Ser. No. 15/348,251, filed on Nov. 10, 2016 which is based on and a claim of priority is made under 35 U.S.C. Section 119(e) to a provisional patent application having Ser. No. 62/415,838 and a filing date of Nov. 1, 2016, and which is incorporated herein by reference.

### BACKGROUND OF THE INVENTION

### Field of the Invention

The present invention generally relates to a healthcare management platform system and method. Specifically, an application server may be implemented to provide access to various user portals of the platform while providing the facility's management with analytical tools such as real-time patient satisfaction.

### Description of the Related Art

Today, online technology and connected platforms support a vast number of options for interconnecting businesses with their clients, such as a hospital with its patients. However, such interconnection does not allow current patients and their guests the opportunity to rate, in real-time, the performance of the hospital's employees while concurrently incentivizing such employees to optimally perform their jobs. A real-time employee rating or survey may alert healthcare facility administrators to current negative situations and allow them to intervene promptly. Accordingly, there is a need in the art to provide a more real-time and user-friendly online interface for the interaction between hospitals and its patients and guests in order to gauge the real-time performance of its employees. Additionally, as will be disclosed, the solution provided by the present invention lends itself to tracking and recording various metrics of patient/guest experiences, which the present invention incorporates further to facilitate analytical review of their interactions, via surveys, with hospital employees.

Further, the present invention may also alert the system to positive interactions between the hospital's employees and current patients and guests via positive online survey scores. Such positive survey scores may automatically translate into points or virtual currency that an employee may redeem for rewards via an online system rewards database. As such, hospitals may leverage the patient generated surveys to promote customer satisfaction by offering employee incentives. In this aspect, positive survey scores, relate to and promote the hospital brand. These types of promotions may typically be combined a contest or rewards as incentives for good work. The benefits of this type of incentive include lower cost of business, direct engagement with its customers, and the receipt of customer surveys that may then be utilized for future planning. The present invention contemplates leveraging these areas together, in the provision of a real-time healthcare platform to provide greater engagement between a hospital and its patients and incentives for employees to optimally perform their jobs.

### SUMMARY OF THE INVENTION

The present invention is generally directed to a healthcare management platform system and method. In initially broad terms, an application server may be implemented to provide a website or application for access to various user portals of the platform, which may include a patient portal, an employee portal, and a administrator portal.

An admitted patient of the healthcare facility may be given access to a private patient portal, via a unique username and password in order to submit surveys based on the performance of the employees of the healthcare facility. In one embodiment, a patient may view his or her health information, condition, and/or recovery plan via the patient portal. An employee may be given access to a private employee portal in order to submit health information of an admitted patient of the healthcare facility. The platform may process employee survey data based on a predefined formula in order to generate employee virtual currency, wherein the application server comprises at least one database in which an employee may redeem the employee virtual currency.

In one embodiment, a patient may designate at least one guest, such that the guest(s) may access the patient portal to submit employee surveys and view the health information of the associated patient. The health information may be written or typed, a voice note, or a picture of video of the patient. The guest(s) may also submit questions to an employee, via the patient portal, regarding the health of the associated patient.

In another embodiment, the guest(s) may install the platform on a remote device, such as a cellular phone. The platform may generate a health alert according to changes in the health information of the patient. The alert may be transmitted over a wireless communication channel to a wireless device associated with the guest(s). This may serve to notify the guest(s) to activate the patient portal to view the health alert. In one embodiment, the health alert may activate the patient portal to cause the health alert to display on the user's remote device whether the remote device is online or offline.

In one embodiment, a patient score and an employee score may be generated by the platform based on a predefined formula(s). The platform may process the patient score with the employee score to generate a patient schedule matrix based on predefined criteria. In one embodiment, the platform may automatically populate the schedule matrix with at least one employee based on a predefined formula. The platform may generate a schedule alert if there are vacancies in the schedule matrix which may be transmitted over a wireless communication channel to a wireless device of an employee(s). In another embodiment, an employee may access the employee portal to search for and submit a request to fill a vacancy in the schedule matrix for which the employee may receive virtual currency, based on a predefined formula of the platform.

In one embodiment, an administrator may be given access to an administrator portal of the healthcare application in order to view employee data such as employee survey data. The platform may notify an administrator regarding negative and/or positive employee survey data based on a predefined formula. An administrator may have access to the administrator portal in which he or she may award discretionary employee virtual currency based on the positive employee survey data. An administrator may access the administrator portal in which he or she may delete employee survey data based on an invalid survey score.

These and other objects, features and advantages of the present invention will become clearer when the drawings as well as the detailed description are taken into consideration.

### BRIEF DESCRIPTION OF THE DRAWINGS

For a fuller understanding of the nature of the present invention, reference should be had to the following detailed description taken in connection with the accompanying drawings in which:

FIG. 1 is a diagrammatic representation of an exemplary system for providing a healthcare management platform of the present invention.

FIG. 2 is an exemplary user interface of a software application for providing a healthcare management platform, illustrating a general patient portal main menu.

FIG. 3 is an exemplary user interface of a software application for providing a healthcare management platform, illustrating a sub-menu featuring healthcare facility departments.

FIG. 4 is an exemplary user interface of a software application for providing a healthcare management platform, illustrating an employee performance survey question.

FIGS. 5A-5B are an exemplary user interface of a software application for providing a healthcare management platform, illustrating an employee portal.

FIG. 6 is an exemplary user interface directed to a software application for providing a healthcare management platform, illustrating a navigation feature of an employee portal.

FIGS. 7A-7B are an exemplary user interface directed to a software application for providing a healthcare management platform, illustrating an administrator portal.

FIG. 8 is an exemplary user interface directed to a software application for providing a healthcare management platform, illustrating an available shifts feature of an administrator portal.

FIG. 9 is an exemplary user interface directed to a software application for providing a healthcare management platform, illustrating a rewards feature of an employee portal.

FIG. 10 is an exemplary user interface directed to a software application for providing a healthcare management platform, illustrating an employee awards feature of an administrator portal.

FIG. 11 is an exemplary user interface directed to a software application for providing a healthcare management platform, illustrating an employee news feed feature of an administrator portal.

FIG. 12 is an exemplary user interface directed to a software application for providing a healthcare management platform, illustrating an employee's feature of an administrator portal.

FIG. 13 is an exemplary user interface directed to a software application for providing a healthcare management platform, illustrating a patient feature of an administrator portal.

FIGS. 14A-14B are an exemplary user interface directed to a software application for providing a healthcare management platform, illustrating a survey results feature of an administrator portal.

FIG. 15 is a flow chart directed to a method of the present invention.

Like reference numerals refer to like parts throughout the several views of the drawings.

### DETAILED DESCRIPTION OF THE PREFERRED EMBODIMENT

As schematically represented in the accompanying drawings, the present invention is generally directed to a healthcare management system and method. Specifically, an application server may be implemented to provide a website or application for access to various users of the platform.

Overview of Implementation System(s)

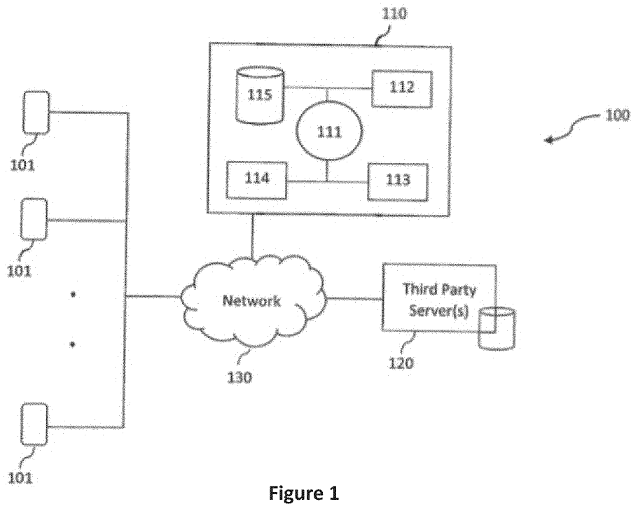

In one embodiment, a healthcare platform of the present invention may be deployed as a software as a service (SaaS) in accordance with implementation on a system 100 as generally represented in FIG. 1. Accordingly, the system 100 of the present invention generally comprises at least one client device 101 communicably connected to an application server 110 over a network 130. One or more third party server(s) 120 may further be communicably connected to the application server 110 and the at least one device 101 over the same network 130.

The device 101 may comprise a mobile device, a tablet, a computer, a wearable electronic device, or any other device or combination of circuits structured and configured to communicate with another device, computer, or server over the network 130. The device 101 may comprise application(s) and user interface(s) (front-end interface) that allow a user to interact with the application server 110 and any third party server(s) 120 and stored applications and programs thereon (back-end processing). The user interface may be proprietary and may comprise a custom developed mobile or desktop application(s). Alternatively, or in addition to, the user interface may comprise a web browser, mobile browser, or other application or executable code that allows for communication and visualization of information.

The term “application server” 110, “third party server” 120 refer to at least one computer having appropriate hardware and applications installed thereon for the provision of server services including web and other functional services described herein, such that a user may access, execute, and/or view the applications remotely from a device 101. More specifically, the application server 110 and third party server(s) 120 may comprise general purpose computers, specialized computers, or other hardware components structured and configured to receive, process, transmit, and store information to and from other devices. The application server 110 is further configured with executable or interpretable computer code that allows it to perform the processes described within this application.

For example, the application server 110 may comprise a general purpose computer comprising a central processing unit (CPU) 111, which may be a single core or multi core processor, memory 114 (random-access memory, read-only memory, and/or flash memory) or primary memory for high-speed storage of executing programs, electronic storage unit 115 (e.g., hard disk) or secondary memory for storing data, communications interface 112 (e.g., network adapter) for communicating with other devices or computers over a network, and/or peripheral device(s) 113 in communication with the CPU 111 that enable input/output of the application server 110.

The application server 110 may implement the methodology of the present invention using any number of solution stacks (a set of software subsystems or components) known to an ordinary computer or web programmer skilled in the art. These solution stacks may include, without limitation, ZEND Server, APACHE Server, NODE.JS, ASP, PHP, Ruby, XAMPP, LAMP, WAMP, MAMP, WISA, LEAP, GLASS, LYME, LYCE, OpenStack, Ganeti, MEAN, MEEN, XRX, and other past, present, or future equivalent solution stacks, or combinations thereof, known to those skilled in the art that allows a programmer to develop the methods and computer programs described within this application. The software stack might be implemented without third-party cloud platforms, for example using load balancing and virtualization software provided by Citrix, Microsoft, VMware, Map-Reduce, Google Filesystem, Xen, memory caching software such as Memcached and Membase, structured storage software such as MySQL, MariaDB, XtraDB, etc. and/or other appropriate platforms. Of course, these solution stacks may also be deployed in cloud platforms by using known development tools and server hosting services such as GitHub and Rackspace, as well as their equivalents.

The third party server(s) 120 may comprise any combination of hardware and software (code segments in any number of programmable, executable, or interpretable languages that support the functionality of the methods described herein) configured to host and transmit items of a user. The third party server(s) 120 may be configured to communicate directly to the application server 110 via application programming interfaces or upon the request of a user.

User account services may be implemented using one or more solution stacks as described above. Alternatively, third party login services such as Facebook, Twitter, LinkedIn, Google and other related services, may be utilized for user account login and authentication, such as via existing third party server(s) 120 of other parties.

The network 130 may comprise at least two computers in communication with each other, which may form a data network such as via LAN, WAN, Serial, Z-WAVE, ZIGBEE, RS-485, MODBUS, BACNET, the Internet, or combinations thereof. The connections may be facilitated over various wired and/or wireless mediums or any combination thereof including interconnections by routers and/or gateways. Network 130 may comprise additional hardware components and/or devices appropriate for facilitating the transmission and communication between the various systems and devices of the present invention, such as those directed to integrated authentication, quality control or to improve content delivery such as via a content delivery network (CDN).

Various aspects of the present invention may be thought of as “products” or “articles of manufacture” typically in the form of machine (or processor) executable code, interpretable code, and/or associated data that is carried on or embodied in a machine readable medium. Machine-executable code can be stored on an electronic storage unit, such memory (e.g., read-only memory, random-access memory, flash memory) or a hard disk, as described above.

All or portions of the software may at times be communicated through the Internet or other communication networks. Such communications, for example, may enable loading of the software from one computer or processor onto another, for example, from a management server or host computer onto the computer platform of an application server, or from an application server onto a client computer or device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, tangible “storage” media, terms such as computer or machine “readable medium”, refer to any medium that participates in providing instructions to a processor for execution. Further, the term “non-transitory” computer readable media includes both volatile and non-volatile media, including RAM. In other words, non-transitory computer media excludes only transitory propagating signals per se, but includes at least register memory, processor cache, RAM, and equivalents thereof.

Therefore, a machine readable medium, such as computer-executable code and/or related data structures, may take many forms, including but not limited to, a tangible storage medium, a carrier wave medium or physical transmission medium. Non-volatile storage media include, for example, optical, magnetic, or solid state disks, such as any of the storage devices in any computer(s) or the like, such as may be used to house the databases. Volatile storage media include dynamic memory, such as main memory of such a computer platform. Tangible transmission media may include coaxial cables, copper wire and fiber optics, communication buses. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

Overview of the Healthcare Management Platform

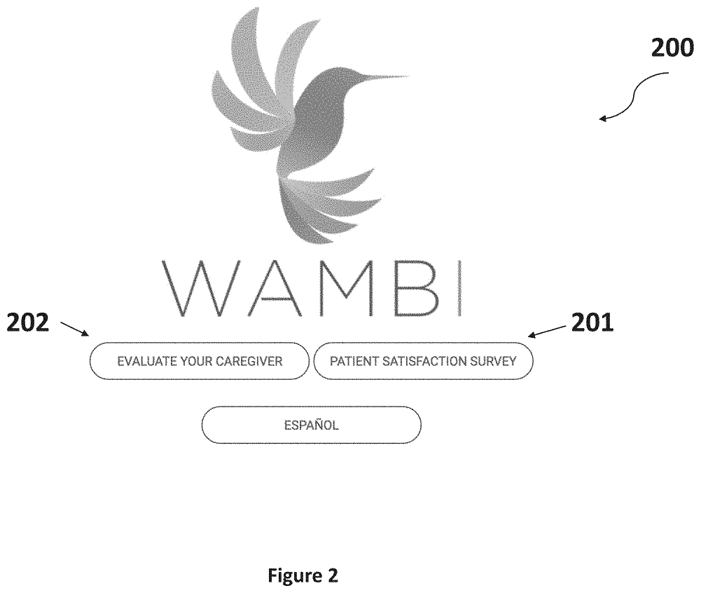

Drawing attention to FIGS. 2-15, embodiments of the present healthcare management platform are illustrated from an end user's perspective. In initially broad terms, the present invention provides a platform or system for the management of a healthcare system, which may further comprise unique real-time patient satisfaction analytical tools, employee rewards functionality, and scheduling procedures of the healthcare system. Additionally, the platform may comprise a plurality of user portals, such as but not limited to, a patient portal, an employee portal, and an administrator portal. Further, each portal may be privately accessed via a unique username and password.

The embodiments illustrated of exemplary user interfaces herein relate to a desktop embodiment, but may similarly be implemented in a mobile or any other operating system known to those skilled in the art. As illustrated at FIG. 2, the platform may comprise a patient portal 200 of the application server 110, accessed by a patient via a unique username and password. A patient may access the application server via a client device 101 such as a mobile device or computer as described above. The unique username and password may allow a patient to enter the patient portal 200 in order to navigate different sections of the program that pertain to the patient only, in order to be compliant with privacy laws.

The patient portal 200 may comprise involving account or user registration information for various online platforms, as known to those skilled in the art. For example, a patient may be first required to register with the application server 110 prior to being able to navigate the program. The registration process may require each patient to input demographic information or other identifying information such as user name, email address, password reminders, communication information, and other appropriate information.

A patient may be able to designate at least one guest to access the patient portal 200 via the patient's username and password. In one embodiment, the platform may associate each designated guest with a patient via a database, or equivalent, as known to those skilled in the art, whereby the designated guest(s) may access the patient portal 200 via a unique username and password. This will serve to allow the guest(s) access to the patient portal 200 but limit the accessible content to only information regarding the patient in which the guest(s) is linked. For example, ‘Patient A’ may designate 5 (five) family members and 1 (friend) as ‘guests’ on the platform, whereby all 6 (six) guests can access the patient portal 200 via a unique username and password or via Patient A's username and password in order to navigate the program as would Patient A.

As illustrated in FIG. 2, the patient portal 200 may initially comprise a simplified main menu 200 whereby a patient/guest may navigate to Take a Survey 201 or Patient Information 202. A patient/guest may navigate through the patient portal 200 by interacting with a select user interface element, such as a button.

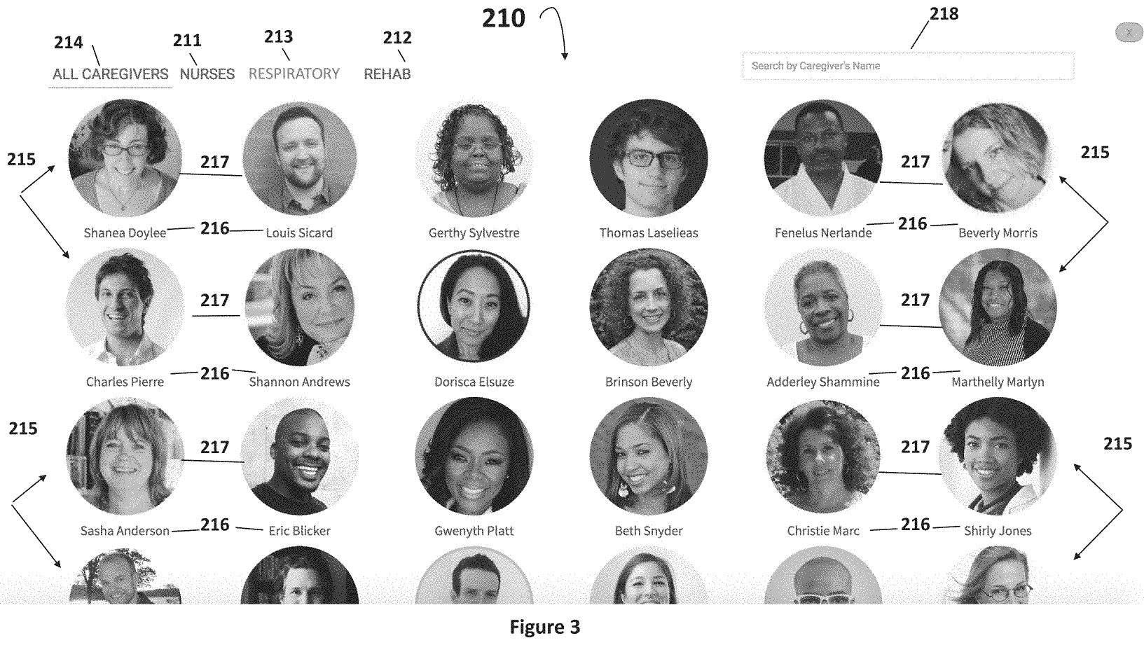

As illustrated in FIG. 3, upon navigating to Take a Survey 201, a patient/guest may be directed to a departments menu 210 in order to submit survey(s) based upon the performance of the employee(s) of the healthcare facility. As discussed in greater detail hereinafter, each patient will be assigned or linked to at least one employee in the platform via a database entry as described above. In one embodiment, the patient/guest may only submit a survey(s) based upon the performance of employee(s) that are linked to the patient in the healthcare management system. In other embodiments, the patient/guest may submit survey(s) based upon the performance of all employees within the system.

A patient/guest may first be directed to the departments menu 210 may comprise a sortable list of at least one healthcare department, provided in scrollable or list view. The departments menu 210 may comprise a sub-menu whereby a patient/guest may further navigate to a particular department within the healthcare facility in order to search for an employee(s). The departments menu 210 may comprise healthcare department(s), such as but not limited to, Nurses 211, Rehab 212, Respiratory 213, and/or All Caregivers 214. Upon selecting a department, a patient/guest may be directed to a populated list of at least one employee profile 215 representing each employee of the selected department as illustrated in FIG. 3. The populated list of employee profiles 215 may be sortable on first name, last name, etc.

Each employee profile 215 may include an employee name 216 and an employee picture 217. The employee picture 217 may be a substantially circular “close-up” picture of the employee's face. This will serve to help a patient/guest locate and identify a particular employee. For example, a patient may not recall certain employees' names, due to the patient's health condition or medication, but may recognize them through their picture on the system. This “facial recognition” may allow a patient/guest to remain anonymous, instead of having to ask an employee for his or her name prior to submitting a survey on that employee. In one embodiment, the present invention may include an automated camera, connected to the time clock system, which may take a picture of an employee each time the employee “punches-in”. This may serve to further help a patient/guest identify an employee who may have changed their appearance or aged since their original picture was taken and saved in the system.

A list of the employee profiles 215 may be provided in a list or scrollable view. In one embodiment, selecting a department from the list of departments menu 210 may navigate the patient/guest to a separate, more detailed page of the program. For example, selecting or clicking the Nurses 211 button may navigate the patient/guest to another page of the program, such as a Nurses 211 sub-page.

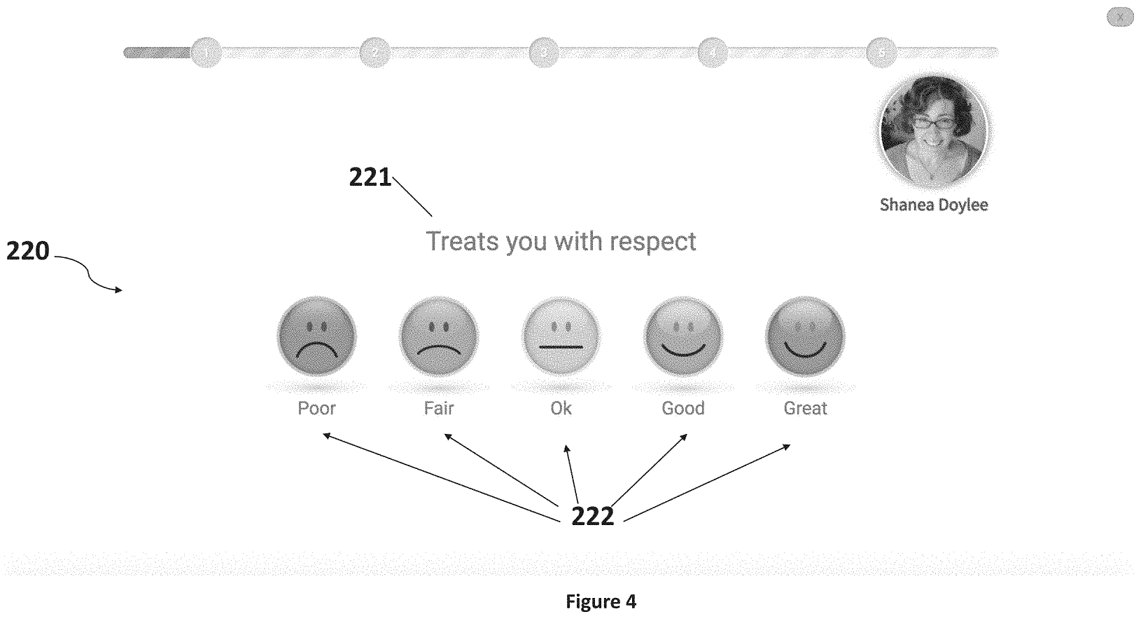

The departments menu 210 may also include an employee search interface 218 to help identify an employee(s), including auto-complete features as is known in the art. For example, a patient/guest may only recall that an employee's name started with a ‘B’. By entering the letter ‘B’ in the employee search interface 218, all employee profiles 215 with a first and/or last name that begins with the letter ‘B’ may “pop-up” on their screen. Upon identifying an employee profile 215, the employee profile 215 may be selectable for survey(s). In one embodiment, only the employee profiles 215 that are associated with the patient, as described above, will be viewable and selectable. Upon selecting an employee profile 215, a patient/guest may be directed to an employee survey 220 associated with the selected employee, as is illustrated in FIG. 4. The employee survey 220 may comprise at least one survey question 221 and a plurality of selectable corresponding survey answers 222. In one embodiment, a plurality of survey answers 222 may initially comprise a high-to-low range, such as, “Great”, “Good”, “OK”, “Fair”, and “Poor”. In another embodiment, the survey answers 222 may comprise a simple mechanism as simple as “Like”, a binary mechanism such as “Like” and “Dislike”, or a point system such as a score from 1 to 10 or other appropriate ranges.

One survey answer 222 may be selected per survey question 221 via a button or interactable element. In one embodiment, a patient/guest may be limited to participating in only one employee survey 220 per employee in a 24 hour period. In other embodiments, a patient/guest may submit a plurality of employee surveys 220 per employee. In another embodiment, an employee may submit a survey(s) on another employee of the healthcare facility. A low survey score(s) may indicate a problem between employees, in which an administrator may have to immediately intervene.

Upon navigating to Patient Information 202, the patient/guest may be directed to a monitoring function of the present invention, in which the patient/guest may monitor health information of the patient, via the patient portal 200. This will serve to allow the designated guest(s) to monitor the patient's health condition and recovery plan remotely, instead of having to visit the healthcare facility or rely on second-hand information for updates. The viewable health information may comprise a predefined list of drop-down items that explain the milestones, based on the patient's condition and recovery plan. In one embodiment, the health information may comprise written or typed information regarding the patient's condition and recovery plan, entered into the system via an employee. In another embodiment, the health information may comprise photographs and/or videos regarding the patient's condition and recovery plan, uploaded to the platform via an employee. In another embodiment, the health information may comprise voice notes or messages regarding the patient's condition and recovery plan, uploaded via an employee.

In at least one embodiment, the patient/guest may ask questions to an employee of the healthcare facility regarding the health information of the patient. As discussed above, the health information may comprise written or typed information, voice notes, and/or video messages via a button or interactable element.

In one embodiment, a guest may install the patient portal 200 on a remote device, such as a cellular phone, tablet, smart watch, computer, etc. in order to access the patient portal 200 from a remote location. Additionally, the guest(s) may request notifications of the patient's health information by entering a wireless device number into the settings function of the present invention. As discussed in more detail hereinafter, the system may generate a health alert if the health information associated with the patient changes. For example, if the condition of the patient worsens, the system may transmit the health alert to the wireless device associated with the guest. The health alert may be transmitted via text message, email notification, push notification, etc. and may accompany a sound effect in order to direct the guest's attention to the wireless device. In one embodiment, a guest may share the health information of the patient via one or more social media platforms known to those skilled in the art, such as via Facebook, Twitter, Instagram, and others.

The health alert may be entirely readable on the wireless device. In another embodiment, the health alert may notify the guest(s) to activate the patient portal 200 on his or her remote device in order to view the health alert in more detail. In one embodiment, the transmitted health alert may activate the installed patient portal 200 to cause the health alert to display on the remote device, whether the patient portal 200 is online or offline. This will serve to instantaneously notify the guest(s) of critical, time-sensitive health information of the patient. For example, if the condition of a patient rapidly deteriorates, the health alert may be instantaneously transmitted to the wireless devices of the guest(s) that are associated with the patient. This instantaneous information may serve to allow the guest(s) more time to rush to healthcare facility than they would have had otherwise.

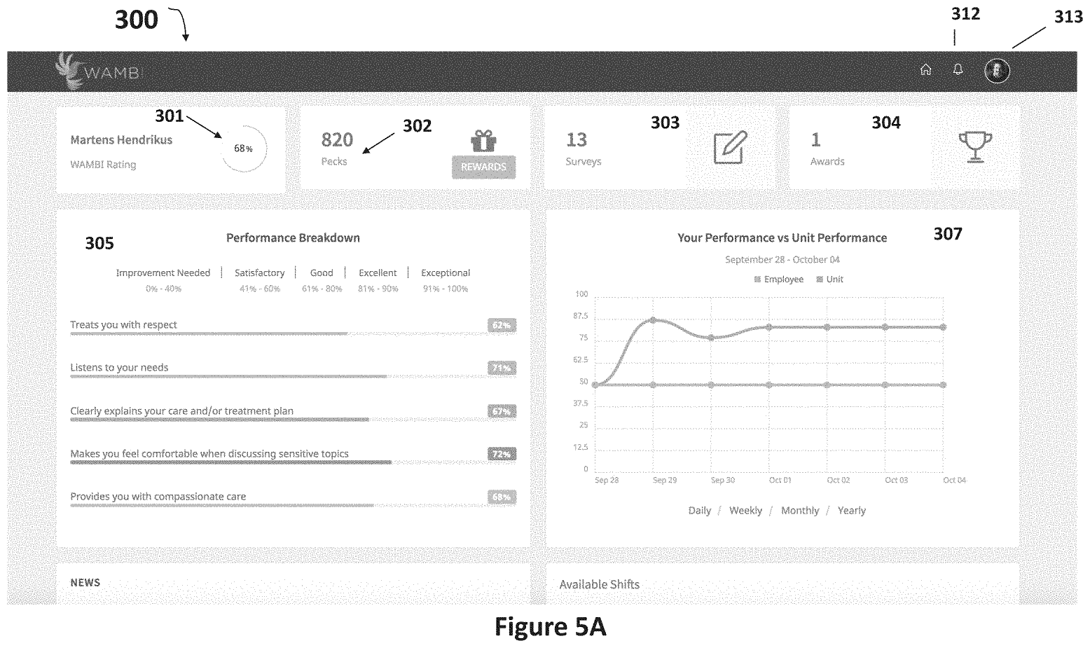

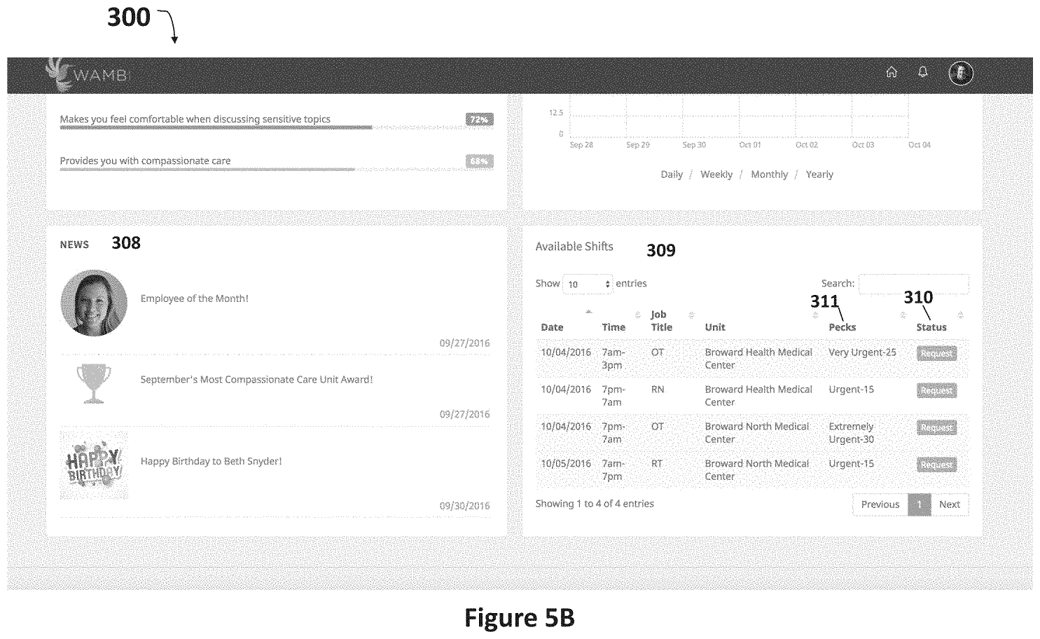

Drawing attention to FIGS. 5A-5B, another exemplary user interface may be the employee portal 300 which may be accessed by an employee of the healthcare facility via a unique username and password. As discussed in detail hereinafter, the unique username and password may allow an employee to enter the employee portal 300 of the application server 110 in order to navigate different sections of the program. An employee may access the application server via a client device 101 such as a mobile device or computer as described above. The employee portal 300 may initially comprise a navigation overlay that may appear upon interaction with a button, allowing an employee to view a plurality of selectable functionalities.

For example, and as discussed in greater detail hereinafter, for each employee portal 300, a “Wambi” employee rating may be illustrated at 301. A Wambi employee rating 301 may indicate an employee's average individual performance rating (for example, via a percentage range from 0%-100%) based on a predefined formula, name, and/or job title.

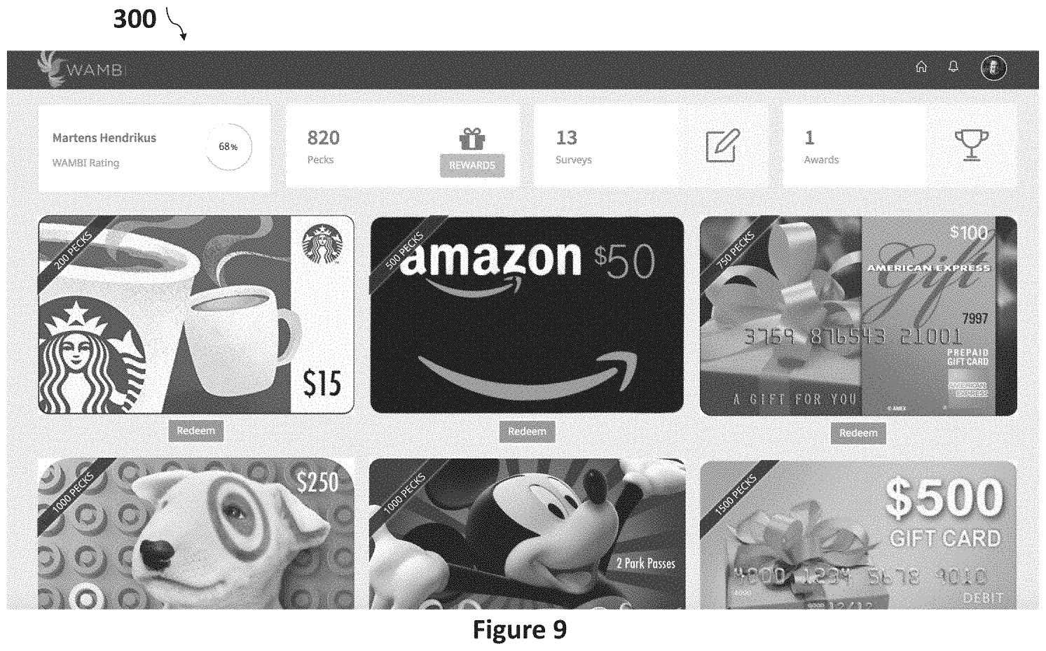

An employee virtual currency amount or “Pecks” may be illustrated at 302 indicating the employee's current employee pecks 302 balance. Employee virtual currency or pecks 302 may be awarded as an incentive to an employee of the healthcare facility upon the performance or meeting of various conditions. In one embodiment, employee pecks 302 may be selectable via a button or other user interface element, in order to navigate the employee to an affiliate rewards system to redeem employee pecks 302 for rewards as illustrated in FIG. 9. An employee may earn employee pecks 302 upon meeting predefined conditions or formulas, such as but not limited to, receiving positive survey scores, filling vacancies in the schedule, acquiring tenure and/or referring other employees to the facility.

Various embodiments of the present invention, as discussed generally above, may comprise predefined procedures, such as but not limited to, mathematical algorithms or formulas, to calculate the Wambi employee rating, department rating, and/or unit rating, as well as Pecks awards.

Accordingly, three formulas or algorithms of the present invention may comprise calculating:

$$A Wambi rating of employees by Individual Wambi Rating \% = 100 \times \frac{Total Pecks earned from surveys}{Total possible Pecks available from surveys} ;$$

$$A Wambi department rating by Department Wambi Rating \% = 100 \times \frac{Total Pecks each department earned from surveys}{\begin{matrix}Total possible pecks available \\ from surveys filled out per discipline\end{matrix}} ;$$

$$A Wambi unit rating by Unit Wambi Rating \% = 100 \times \frac{Total pecks each unit earned from surveys}{Total possible pecks available from surveys filled out per unit } .$$

For purposes of the above formulas, survey answers 222 may be represented as ordinal data, for example, from “Poor” to “Great”. Further, Peck values may be determined by the empirical rule, as known in the art. The platform of the present invention may automatically determine if employee surveys 220 are positively or negatively skewed in comparison to the originally determined “normal” distribution, and may automatically adjust the Peck values for each employee survey 220 to further enable a normal or forced distribution. A normal distribution may help identify the top and bottom survey scoring employees, while still motivating the employees who are considered ‘average’, falling within two standard deviations of the mean. This may serve to create a relative scale for employee ‘shaping’ processes, help achieve organizational goals and gain meaningful transparency on employee performance. As such, the necessary focuses may be recognizing the top scoring employees and strategically training the lower scoring employees.

The platform may assign Pecks to each employee survey 220, which may be provided to the employee as well as the administrator(s) in real-time. The formula or algorithm places more emphasis, higher Peck values, on both the top two survey responses (ex. “Good” and “Great”) and places less emphasis, lower Peck values, on the bottom two survey responses (ex. “Poor” and “Fair”). This will serve to provide more of a difference between the “best” and “worst” employees. Further, the majority of employees that perform on an “average” level may be still validated because they may receive just enough to stay motivated, but not too much to become discouraged and avoid striving for improvement.

In a “normal” distribution, 68% of the data may fall between 1 standard deviation of the mean, 95% of the data may fall between 2 standard deviations of the mean, and 99.7% may fall within 3 standard deviations of the mean. The present invention may initially determine the mean performance score based on patient experience data from the general population, depending on the type of unit or the region etc. However, over time, the platform may learn the particular unit's mean and may automatically adjust the Peck values for each survey answer 222 to create a ‘forced normal’ distribution, heavily awarding the employees in the top 50%, who may be 2-3 standard deviations from the mean and minimally awarding the employees in the bottom 50%, who may be 2-3 standard deviations lower than the mean. Within this forced distribution, the goal may be to validate employees who are in the top 50% of employee surveys 220, for example, receiving an overall average of “Ok”, “Good”, or “Great” Wambi Rating 301. Consequently, employees who score in the bottom 50% may encompass the “Ok”, “Fair”, or “Poor” Wambi Rating 301. Further, the present invention minimalized the difference in Peck values between a “Good” and “Great” employee because scoring in the ‘tail end’ of the distribution by achieving all “Great” responses on an employee survey 220 may be too infrequent. Additionally, low peck values may be assigned to “Poor” or “Poor/Fair” employee surveys 220.

As discussed in greater detail below, an employee survey breakdown 305 may serve to ensure that employees, as well as administrators, understand the percentages in regards to organizational standards and expectations. As such, based on a forced distribution and subject to an automatic change, when a unit's mean performance is determined, there may be at least 5 categories with percentage ranges that easily assign an interpretable meaning to the real-time survey results to be used by employees and administrators.

For example, Wambi Ratings 301 (for individuals, per department and per unit) of: 0%-59% may indicate ‘Improvement needed’; 60-69% may indicate a ‘Satisfactory’ performance; 70-79% may indicate a ‘Good’ performance; 80-89% may indicate an ‘Excellent’ performance; and 90-100% may indicate an ‘Exceptional’ performance. These 5 categories may be determined based on a negatively skewed distribution that is forced into a normal distribution based on weighted Peck values.

Accordingly, Peck values for “Good” average survey results may be 83.33% of an average “Great” score value; “Ok” average results may be 50% of an average “Great” score value; “Fair” average results may be 16.67% of an average “Great” score value; and “Poor” average results may be weighted as 0%. This weighting scale may keep the employees who score within the first standard deviation of the mean ‘motivated’. The difference between “Ok” average results and “Good” average results may require employees to put forth more effort to elevate their score in order to surpass “Ok” results. However, once they surpass “Ok” results, up to an average score of “Good”, they may receive an average jump of a 33.33% increase in Pecks per survey, which may be more rewarding than a 25% increase, which would exist with equal-ordinal data values. The platform may recognize employees who score within 2 and 3 standard deviations of the mean for the top 50% of employees and may distinguish this difference more so in the Wambi Rating 301 as it may be sorted from highest to lowest based on customizable sorting features, for example, by department. This Wambi Rating 301 may be used as an un-biased method to evaluate employees during performance appraisals.

As discussed in greater detail below, the employee/unit performance graphs 307, each may represent the average percentage score on each survey question 221. This percentage may be calculated by the accumulation of Pecks on each survey divided by the total possible Pecks to be earned on each survey multiplied by 100. The employee/unit performance graphs 307 may utilize the Wambi Ratings 301 of the individual Wambi Rating 301 on a line-graph compared with the Unit or Facility's Wambi Rating 301 over a period of time. This may serve to provide employees and administrators with a easily interpretable graph of comparable performance. However, it is important to be noted that each employee's performance is only visible to that particular employee and administrators. Employees are not able to view other employee's performances.

Employee total surveys may be illustrated at 303 indicating the total amount of performance surveys submitted for the employee. Employee total rewards may be illustrated at 304 indicating the total amount of rewards the employee has earned. An employee survey breakdown 305 may comprise at least one employee survey question, and each survey question further illustrated by an average score percentile for each question, based on a predefined formula.

An employee/unit performance chart or graph may be illustrated at 307 comparing the employee's rating 301 with the overall unit or facility rating. In one embodiment, the employee/unit performance chart 307 may be adjustably based over a time period (for example, by day, week, month, year, etc.).

An employee newsfeed may be illustrated at 308, wherein an employee may view personal information, such as quality-measure rate accomplishments, and/or public information, such as upcoming events, contests, news stories, and orientation and competency requests. In one embodiment, an employee newsfeed 308 may be read-only. In another embodiment, social network features may be implemented to the application server of the present platform, which may allow employees of the platform to “follow” one another, communicate with one another, and comment on each other's “page”. One embodiment of the present invention may comprise a leaderboard list, which may include a list of the top employees with the highest awarded employee pecks or awards for a given time period. In another embodiment, employee may share the leaderboard list across various social platforms.

An employee available shifts function may be illustrated at 309. This interactable function may allow an employee to search for and request available shifts via a button or other interactable element. In one embodiment, employee available shifts 309 may comprise an available shift status 310 button, indicating the current availability of a shift. For example, the available shift status 310 may initially comprise ‘Request’ indicating the shift is available to an employee. Upon click through or selection, the available shift status 310 may change to ‘Pending’ indicating that the employee's request is pending on approval by an administrator. Upon acceptance by an administrator, the available shift status 310 may change to ‘Accepted’ indicating that the employee's request has been approved. Upon denial by an administrator, the available shift status 310 may change to ‘Taken’ indicating that the employee's request has not been approved. Upon cancellation by an administrator of the system, the available shift status 310 may change to ‘Canceled’ indicating that the employee's shift has been cancelled. In one embodiment, the available shift status 310 button may change colors or fonts when the status changes, in order to alert the employee to the change.

In another embodiment, employee available shifts 309 may comprise an available shift pecks 311 award, indicating the amount of virtual currency an employee may be awarded upon completion of an available shift. In one embodiment, an employee may submit preferences, such as for example, preferred shifts and/or the type of patients with which the employee prefers to work. In another embodiment, an employee may view his or her current or future work schedule. In one embodiment, the employee may request vacation days and/or sick days via a button or interactable element of the employee portal 300.

In at least one embodiment, an employee may enter his or her wireless device number and/or an email address into the employee portal 300. As discussed in more detail hereinafter, the healthcare application may generate a schedule alert if there are vacancies or shifts that need to be filled by at least one employee. Upon a schedule vacancy, the system may transmit a schedule alert to at least one employee's wireless device and/or email address. An employee may receive the schedule alert by means of text message, email notification, push notification, and/or other notification methods from a mobile or computing device known to those skilled in the art. In one embodiment, the schedule alert may notify an employee to access the employee portal 300 in order to search for the vacancy and request the shift.

In at least one embodiment, an employee may be directed to an employee/patient function of the employee portal 300, in which an employee may submit health information regarding a patient's condition and recovery plan. This will serve to cause the health information of the corresponding patient to update instantly on the patient portal 200 of present invention. As discussed above, the health information may comprise written or typed information, photographs, videos, and/or voice messages regarding the patient's condition and recovery plan. As mentioned above, upon employee submission of the updated health information, the system may generate and transmit a health alert to the designated guest(s) of the corresponding patient.

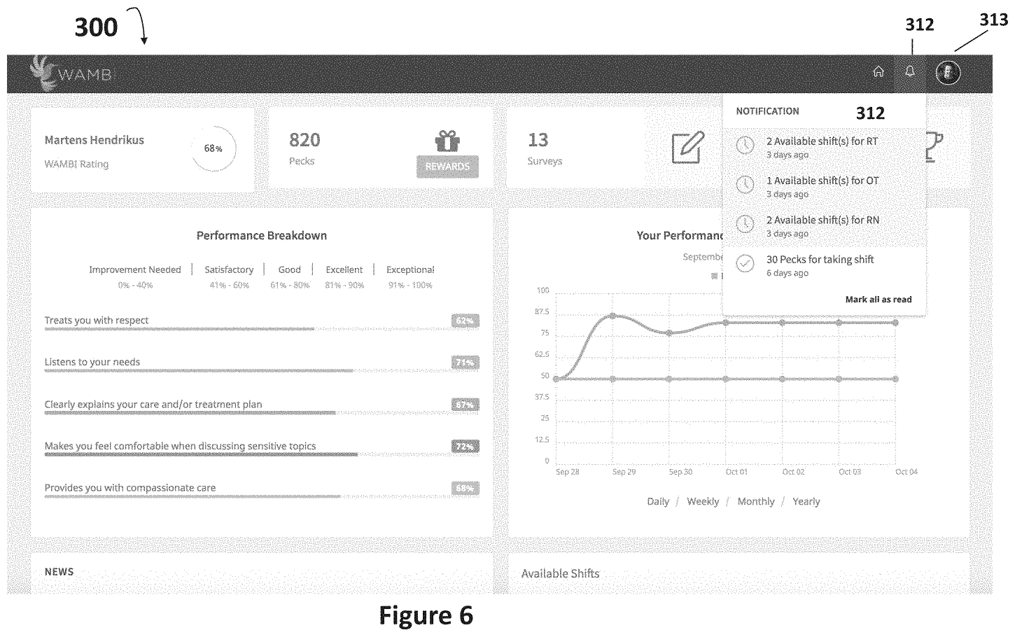

Drawing attention to FIG. 6, an employee notification function may be illustrated at 312 comprising a plurality of notices sent to the employee via the system. The employee notification 312 may initially comprise notifying an employee upon receiving pecks for completing a vacant shift, receiving a specific award, or positive survey results, as well as for available shifts and corresponding status changes. In one embodiment, an employee picture may be illustrated at 313.

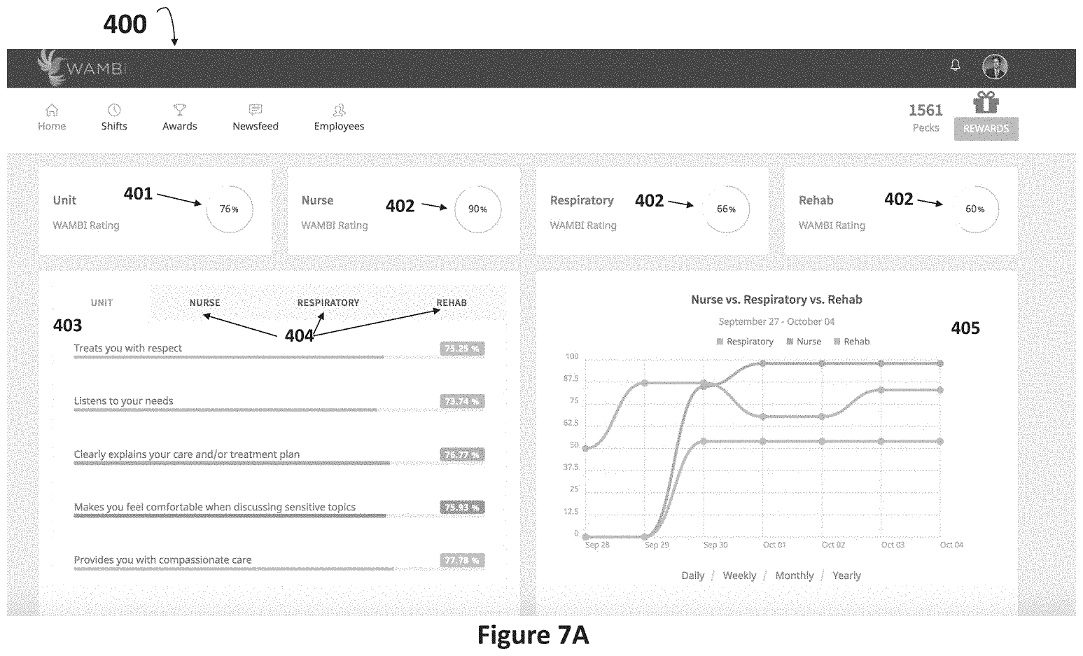

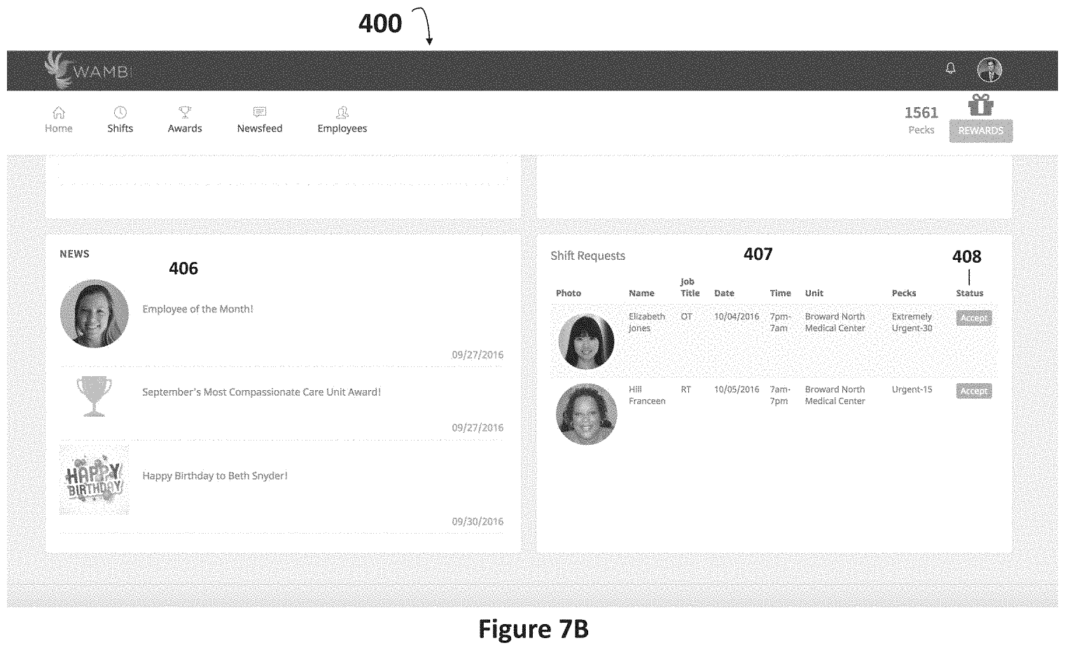

Drawing attention to FIG. 7, another exemplary user interface may be the administrator portal 400 of the application server 110, which may be accessed by an administrator of the healthcare facility via a unique username and password from the application server 110. An administrator may access the application server 110 via a client device 101 such as a mobile device or computer as described above. In one embodiment, all administrators of the healthcare facility may access the administrator portal 400 via a group username and password. As discussed in detail hereinafter, a unique username and password will allow the administrator to enter the administrator portal 400 in order to navigate different sections of the program. The administrator portal 400 may initially comprise a navigation overlay that may appear upon interaction with a button, allowing an administrator to view a plurality of selectable functionalities.

For example, and as discussed in greater detail hereinafter, for each administrator portal 400, a unit or facility rating may be illustrated at 401. A unit rating 401 may indicate a unit's average performance rating of all employees whom work in the unit (for example, via a percentage range from 0%-100%) based on a predefined formula set by an administrator.

A unit may further comprise at least one department. Accordingly, a department rating may be illustrated at 402. A department rating 402 may indicate a specific department's average performance rating of all employees whom work in the department (for example, the “Nurse” department, the “Respiratory” department, and the “Rehab” department illustrated in FIG. 7) based on a predefined formula set by an administrator.

At unit survey breakdown 403 may comprise at least one survey question, and each survey question further illustrated by an average score percentile, based on a predefined formula set by an administrator. A survey question may be uniform for all employees, for example the survey question 221 as discussed above, or may be a select amount or combination of survey questions. At least one department survey breakdown 404 may comprise at least one survey question, and each survey question further illustrated by an average score percentile, based on a predefined formula set by an administrator. The unit survey breakdown 403 and the department survey breakdown 404 may comprise selectable tabs (for example, the “Unit” tab, the “Nurse” tab, the “Respiratory” tab, and the “Rehab” tab illustrated in FIG. 7) as known to those skilled in the art.

A department/unit performance chart or graph may be illustrated at 405 comparing the various departments' ratings 402 or comparing a department's rating 402 with the overall unit or facility rating 401. In one embodiment, the department/unit performance chart 405 may be adjustably based over a time period (for example, by day, week, month, year, etc.).

A unit newsfeed may be illustrated at 406, wherein an administrator may post information from his/her administrator portal 400 that feeds directly to the employee(s) newsfeed 308, as known to those skilled in the art. For example, an administrator may post personal information to a particular employee, such as quality-measure rate accomplishments, and/or public information to a plurality of employees, such as upcoming events, contests, leaderboard lists, news stories, and orientation and competency requests.

A shift request function may be illustrated at 407. This interactable function may allow an administrator to accept or deny employee requests for available shifts via a shift request status 408 button(s), wherein the system may instantaneously update an employee(s) portal 300 to alert the employee(s) that his/her shift request was accepted or denied, as known to those skilled in the art.

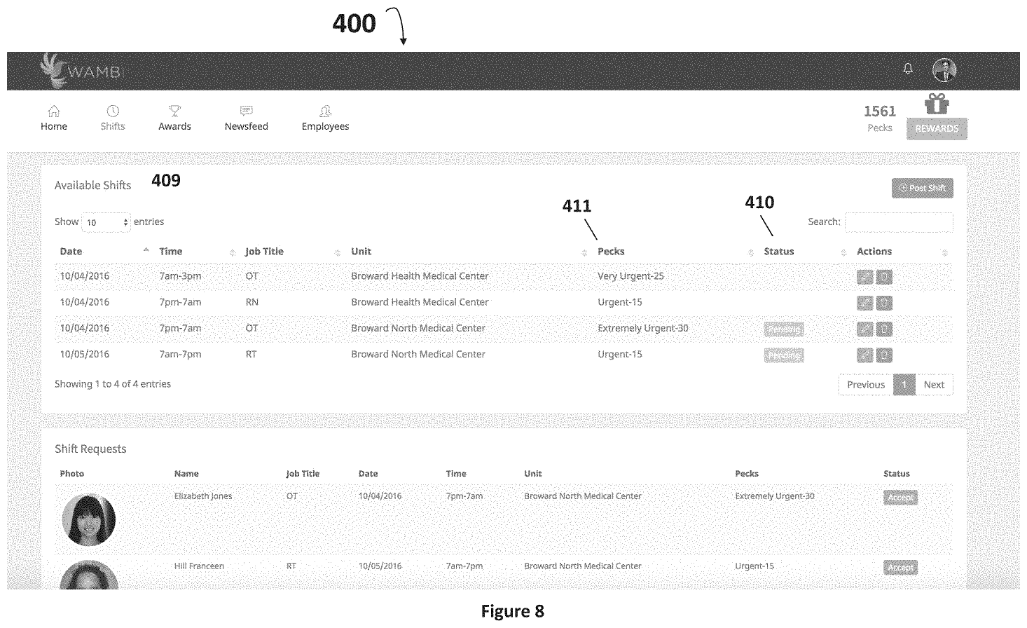

As illustrated in FIG. 8, the administrator portal 400 may comprise an available shift function 409, wherein an administrator may add/modify/delete available shifts. The available shift function 409 may include an available shift status 410 button(s), indicating the current availability of an available shift. For example, the available shift status 410 may initially comprise ‘Request’ indicating the shift is available to employees. Upon request of an available shift by an employee, the available shift status 410 may change to ‘Pending’ indicating that an employee has requested the shift and is pending on approval by an administrator. Upon acceptance by an administrator, the available shift status 410 may change to ‘Accepted’ indicating that an employee's request has been approved. Upon denial by an administrator of an employee's request, the available shift status 410 may change back to ‘Request’ indicating that the shift is available. Upon cancellation by an administrator, the available shift status 410 may change from ‘Accepted’ to ‘Request’ indicating that the shift is available. In one embodiment, the available shift status 410 button may change colors or fonts when the status changes, in order to alert an administrator to the change. The available shift function 409 may also include an available shift pecks 411 award, indicating the amount of virtual currency an employee may be awarded upon completion of an available shift.

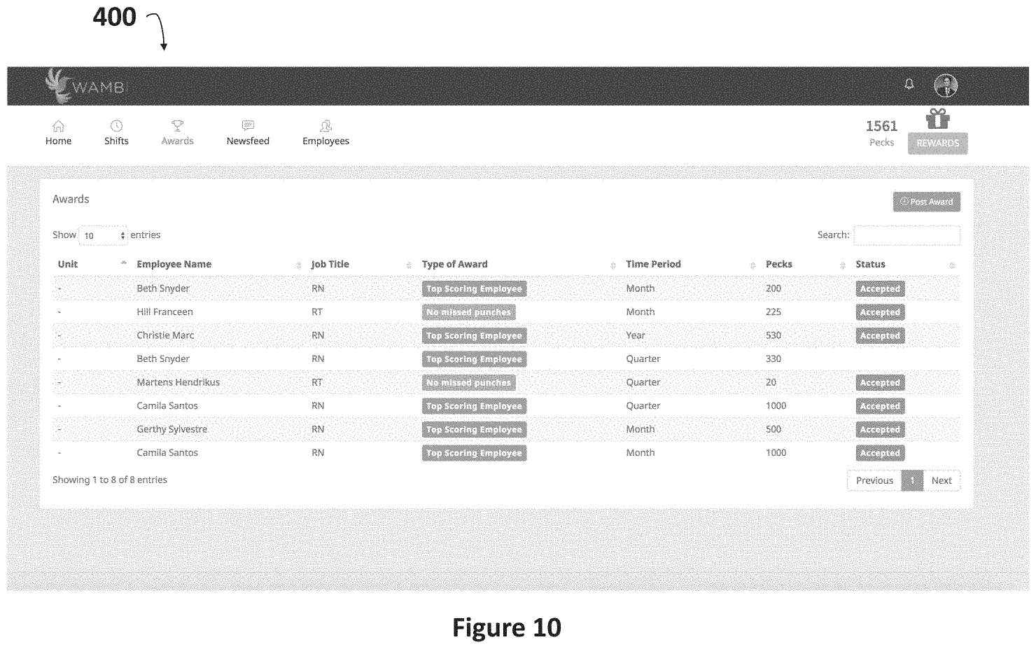

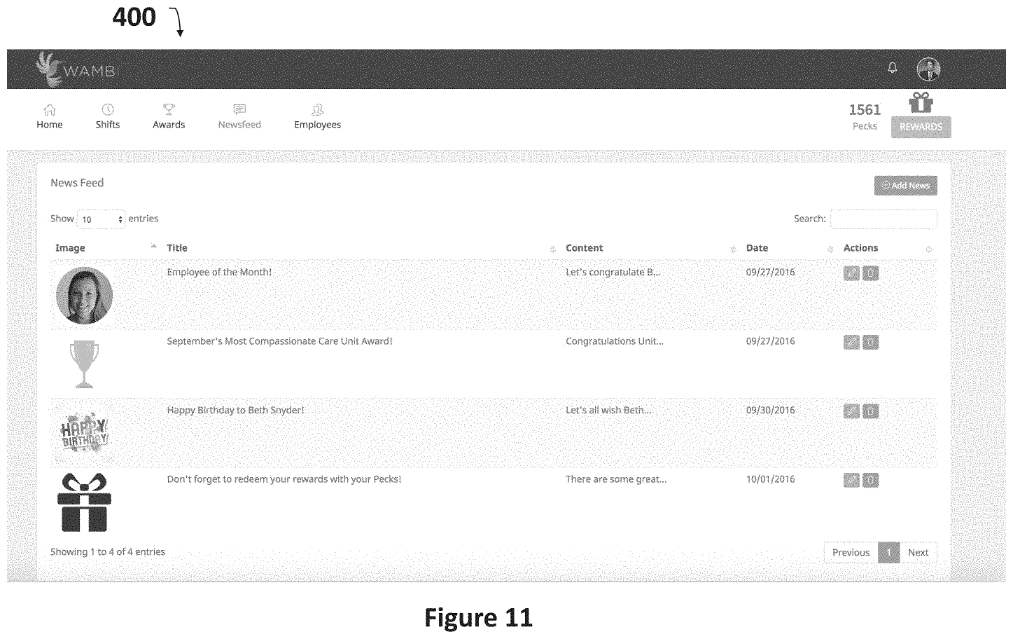

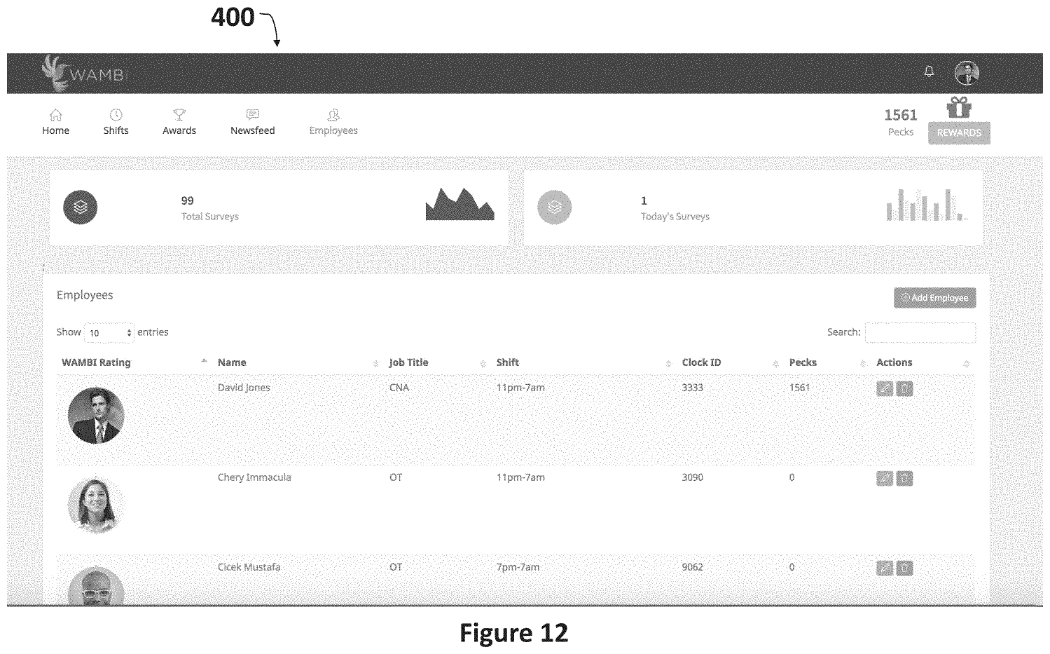

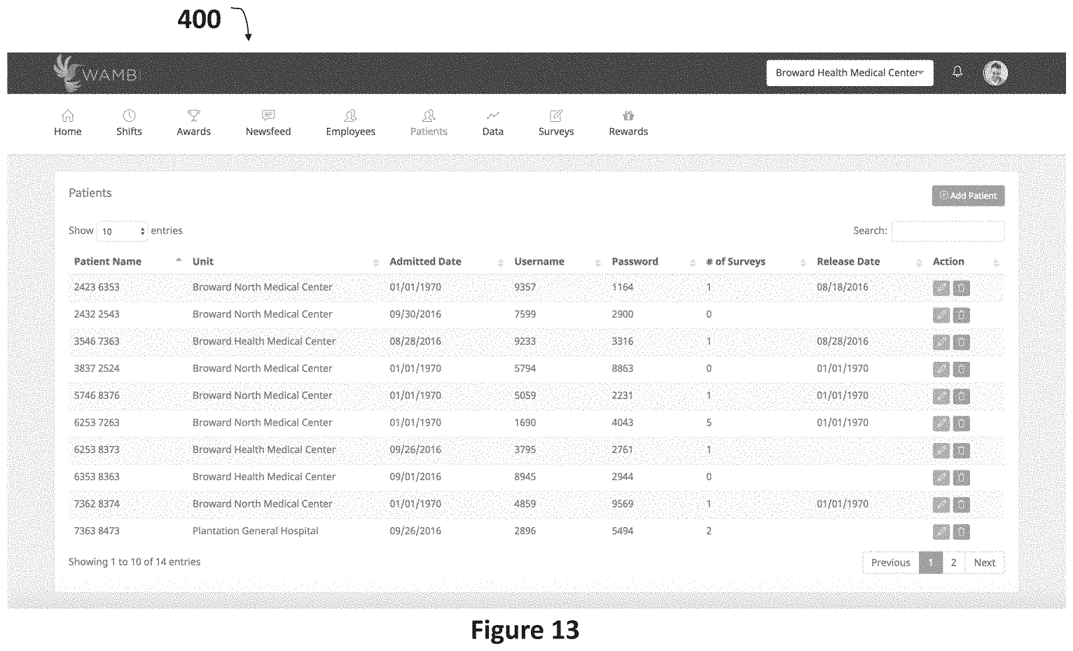

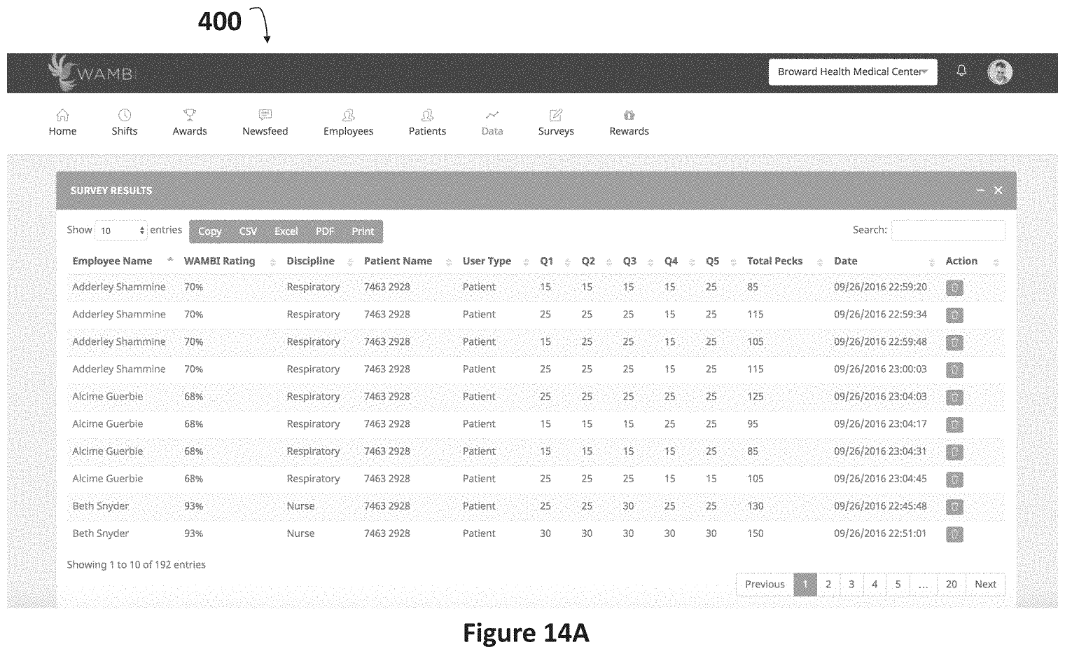

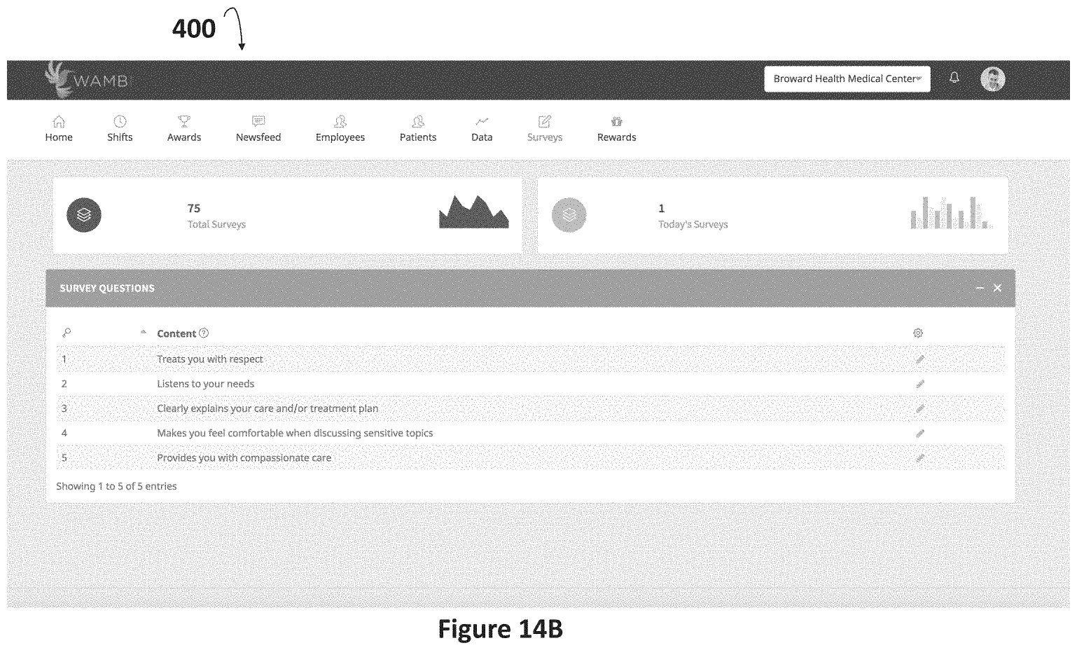

An administrator may display employee rewards and post new awards as illustrated at FIG. 10. An administrator may post information that may populate the employee newsfeed as illustrated at FIG. 11. An administrator may view current employee data, add new employees, and/or delete former employees as illustrated at FIG. 12. An administrator may view current patient data, add new patients, and/or delete former patients as illustrated at FIG. 13. Employee survey data may be viewable and sortable as illustrated at FIGS. 14A-14B.

In one embodiment, the data columns of some the above Figures may be broken down by ‘employee performance data’, ‘patient experience data’, and/or ‘unit performance data’. This data may have multiple sorting features and may be exportable into multiple formats (i.e., excel). The key sorting features may be sorting by Wambi Rating (individual, discipline, or unit) 301, as well as by ‘patient insurance payor source’. This relevant information may serve to be presented to insurance companies when negotiating rates for reimbursements. The Wambi Rating for their employees, their patient experiences, as well as for their unit as a whole, may be sorted by insurance payor source so that high ratings can be leveraged for supporting data analytics in negotiations.

However, it is emphasized that at least some of the above features, illustrated by the Figures, may only be accessed and/or modified by some administrators or managers of the system, depending on their security level. For example, “administrators” in one facility may be divided by areas, such as but not limited to, “Patient Managers”, “Employee Managers”, and/or “Scheduling Managers”. In another facility, “administrators” may be divided between “Administrators” and “Super Administrators” etc.

In one embodiment, an administrator may view the employee pecks 302 of an individual employee or a plurality of employees combined as a unit, facility, region etc. This view may show the number of pecks an employee(s) has available, the total number of pecks generated overall, pecks awarded by category (for example, the pecks awarded via positive survey scores submitted by patients and/or guests), and pecks earnings over a time period (for example, by day, week, month, year, or other time period adjustable by the administrator). An administrator may enter or update the formulas or algorithms which calculate the various employees' Pecks awards etc. In another embodiment, an administrator may award employee pecks on a discretionary basis. In one embodiment, an administrator may award discretionary employee pecks only upon the approval of a super administrator.

In one embodiment, an administrator may view the employee survey results via customizable reports. An administrator may view the data of an individual employee or a plurality of employees combined as a unit, facility, region etc. This may further be broken down by each survey question submitted. This view may further display survey data over a time period (for example, such as by day, week, month, year, or other time period adjustable by an administrator).

The system may generate an alert to the administrator(s) of the platform indicating that an employee has received a plurality of surveys in a period of time. For example, an employee may receive 3 surveys in a 24 hour period, which may prompt the system to notify the administrators to possible suspicious activity, such as an employee wrongfully filling out surveys on themselves. Additionally, a patient/guest may be limited in submitting surveys on a particular employee in a period of time. For example, a patient may be limited to submitting 3 surveys on an employee in a 24 hour period, which may prompt the system to notify the administrators to possible suspicious activity.

The system may generate a survey alert to the administrator(s) of the platform indicating low survey scores based upon the predefined formulas described above. A low survey score(s) may indicate a problem between the patient/guest and an employee(s), in which an administrator may have to immediately intervene. However, a low survey score may also indicate a problem with the patient/guest. For example, if one particular patient submits low ratings, such as “Poor” for every question for 3 employees, the scores may be automatically deleted from the system. This may serve to objectively delete scores instead of leaving the deletion up to an administrator.

Further, the system may generate an alert to the administrators indicating that an employee, who has received only high survey scores in the past, received a low survey score. For example, an employee who consistently receives “Good” and “Great” scores, may prompt an alert if they receive a “Poor” score. The alert may notify the administrators so they can address the situation before it progresses. In one embodiment, the system may generate a survey alert to the administrator(s) for high survey scores based upon a predefined formula. In another embodiment, the administrator(s) may override invalid survey scores based upon predefined criteria.

In one embodiment, an administrator of the platform may input and submit a schedule matrix for each patient. The schedule matrix may initially comprise a predefined ideal employee/patient ratio, whereby an administrator may assign at least one employee per patient, depending upon the health information of the patient. A patient admitted to the healthcare facility in a serious condition may be assigned more employees in the schedule matrix than a patient admitted in a more stable condition. The patient's health information may initially comprise intake or admission prognosis. For example, a patient may be initially diagnosed by an intake doctor or nurse manager as suffering from cardiac arrest, whereby an employee will submit as health information into the platform. In turn, the platform may create an initial employee/patient matrix according to the predefined formula for “cardiac arrest”.

In one embodiment, the patient health information may be calculated to generate a patient score based on a predefined formula such as a point system, whereby health information is pre-scored, such as from 1 to 10, in which a higher score may be indicative of a more serious condition. For example, the patient admitted to the healthcare facility with cardiac arrest may be initially assigned a score of ‘8’, whereby the patient may be assigned 3 (three) nurses at all times. In another embodiment, the patient score may change as the health information of the patient changes. For example, the health information of the patient originally admitted for cardiac arrest may improve and his patient score may update from a score of ‘8’ to a score of ‘3’, whereby the patient may be assigned 1 (one) rehabilitation specialist. As discussed above, the patient health information may be updated by employee(s) via the employee portal 300.

In one embodiment, the patient health information may include existing electronic medical records automatically drawn from the system and combined with the initial patient information to calculate the patient score. For example, the medical records of the patient admitted with cardiac arrest may show that he or she is allergic to morphine. Accordingly, based on a predefined formula, the patient score may change from an ‘8’ to an ‘8A’ indicating the patient may need to be assigned 1 (one) anesthesia specialist in addition to the 3 (three) nurses.

In one embodiment, an administrator may assign at least one employee to a patient via the platform. Upon creation of a new schedule matrix, each opening or cell of the matrix may be empty and/or comprise an add employee button for an administrator to populate each cell with an employee. An administrator and/or the platform may be able to view or display employee timecard data in order to prioritize ‘non-overtime’ employees over ‘overtime’ employees for the current time period.

In some embodiments, the platform of the present invention may comprise a recommendation feature, whereby creating a prioritized list of employees in order for an administrator to fill the cell(s) of a schedule matrix, based on predefined formulas. For example, an administrator may elect via an interface of the present platform to create a new schedule matrix 301 for ‘Patient A’ who suffers from cardiac arrest. The administrator may elect a “Recommend me Employees” option, and a backend rules engine of the application server may auto-populate the cells of the schedule matrix. Each cell of the schedule matrix may be populated with, for example, the highest rated employee(s), the employees who have received positive survey results from past cardiac arrest patients and/or their guest(s), the employees who have submitted a preference to work with cardiac arrest patients, etc. In another embodiment, the platform of the present invention may be able to automatically create a schedule matrix and/or auto-populate the schedule matrix with the required employees based on predefined formulas as described above.

In one embodiment, after the creation of a schedule matrix, an administrator may send or share the matrix with other administrators of the platform or sent to a super administrator to be approved. The approved schedule matrix may be saved on the platform of the present invention, and may be viewable to employees via the employee portal 300. An approved schedule matrix may be edited by an administrator or super administrator at any time. In one embodiment, a sharing administrator may highlight specific cells in the proposed matrix, such as to call attention to them, for example to indicate at least one employee of the proposed matrix will be ‘overtime’ in the current time period if selected. The recipient administrator may edit and/or modify the cells of the matrix, and then pass it back to the sharing administrator or to another administrator.

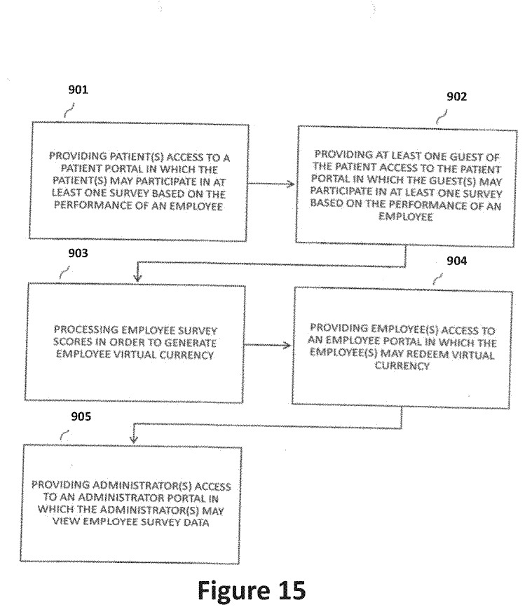

Drawing attention to FIG. 15, a method of the present invention may comprise providing patients(s) access to a patient portal of the platform, which the patients(s) may access via a unique username and password from an application server, as in 901. The patients(s) may participate in at least one survey based on the performance of employees of the healthcare facility. In some embodiments, a patient may access the patient portal to view his or her health information, such as a health condition or recovery plan. In other embodiments, a patient may access the patient portal to submit questions regarding his or her health information to employees of the platform. A patient may access the application server via a client device such as a mobile device or computer as described above. A patient may access the application server via a mobile application interface installed on the client device, or via a browser accessing the application server as a software-as-a-service or equivalent as known to those in the art.

Next, optionally, at 902, the method of the present invention may comprise providing at least one guest(s) of a patient access to the patient portal, wherein the guest(s) may access the patient portal via the patient's username and password or a unique username and password from an application server. The guest(s) may participate in at least one survey based on the performance of employees of the healthcare facility. In some embodiments, a guest(s) may access the patient portal to view the health information of the patient, such as a health condition or recovery plan. In other embodiments, guest(s) may access the patient portal to submit questions regarding the patient's health information to employees of the platform. A guest(s) may access the application server via a client device such as a mobile device or computer as described above. A guest(s) may access the application server via a mobile application interface installed on the client device, or via a browser accessing the application server as a software-as-a-service or equivalent as known to those in the art.

Upon submission of an employee survey, survey scores are processed to generate employee virtual currency automatically, as in 903, at the application server. The employee virtual currency may be generated based on a predefined formula, such as receiving more positive than negative survey scores. In another embodiment, employee(s) may redeem virtual currency at an affiliate rewards system of the application server.

Next, in 904, the method of the present invention may comprise providing employee(s) access to an employee portal of the platform, which the employee(s) may access via a unique username and password from an application server. The employee(s) may access the employee portal in order to view and submit health information regarding the patient(s) of the healthcare facility. In some embodiments, an employee may access the employee portal in order to answer health related questions of a patient, such as from the patient or his or her guest(s). In other embodiments, an employee may access the employee portal in order to search for and request available shifts. Employee(s) may be notified after their request is approved or rejected at the application server. In one embodiment, an employee may access the employee portal in order to view personal information such as virtual currency awards, employee rating, and public information via an employee newsfeed. An employee may access the application server via a client device such as a mobile device or computer as described above. An employee may access the application server via a mobile application interface installed on the client device, or via a browser accessing the application server as a software-as-a-service or equivalent as known to those in the art.

Next, in 905, the method of the present invention may comprise providing administrators(s) access to an administrator portal of the platform, which administrators(s) may access via a unique username and password from an application server. The administrators(s) may access the administrator portal in order to view employee survey information submitted by the patient(s) and/or guest(s) of patient(s) of the healthcare facility. In one embodiment, a system generated alert may be transmitted to an administrator via the portal for low employee survey scores in order to alert an administrator to a possible conflict between a patient and/or guest and an employee. In other embodiments, an administrator may access the administrator portal in order to post available shifts and accept or reject employee requests to fill the available shift. In one embodiment, an administrator may access the administrator portal in order to view employee ratings, department ratings, and/or unit ratings. An administrator may access the application server via a client device such as a mobile device or computer as described above. An administrator may access the application server via a mobile application interface installed on the client device, or via a browser accessing the application server as a software-as-a-service or equivalent as known to those in the art.

Individual components or elements of the system and method may be used interchangeably. The order of the method or processes described above may be arranged in any combination in various embodiments. In some embodiments, various steps may be omitted.

Since many modifications, variations and changes in detail can be made to the described preferred embodiment of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A method for providing a healthcare management application for user(s) of an application server comprising:
configuring a healthcare management platform to include a patient portal and an employee portal;
providing at least one employee access to the employee portal for submission of health data associated for at least one patient;
providing at least one patient access to the patient portal for viewing the health data and for submission of employee survey data of at least one employee;
associating at least one user with the at least one patient;
processing each employee survey datum for the generation of a normally distributed value therefrom;
converting the normally distributed value to a forced value according to a mean value, wherein the mean value is learned from an aggregate of each previously processed employee survey datum;
determining an amount of virtual currency according to the forced value; and
disposing the virtual currency on the employee portal for the redemption thereof by the at least one employee via at least one interconnected database.

2. The method of claim 1 wherein the healthcare management platform is further configured to include an administrator portal disposed for access of the employee survey data by at least one administrator.

3. The method of claim 1 wherein the health data may comprise typed information.

4. The method of claim 1 wherein the health data may comprise at least one voice message.

5. The method of claim 1 wherein the health data may comprise at least one photograph or video.

6. The method of claim 1 further comprising:
providing the at least one user access to the patient portal in which the at least one user may submit questions regarding the health data associated with the patient.

7. The method of claim 6 wherein the questions may comprise typed information.

8. The method of claim 6 wherein the questions may comprise at least one voice message.

9. The method of claim 1 further comprising:
installing the patient portal on a remote wireless device associated with the at least one user;
generating a health alert determinative of changes in the health data associated with the at least one patient;
transmitting the health alert over a wireless communication channel to the wireless remote device associated with the at least one user; and
notifying the at least one user to activate the patient portal of the healthcare application to view the health alert.

10. The method of claim 9 wherein transmitting the health alert comprises activating the patient portal causing display of the health alert on the wireless remote device whether the remote device is online or offline.

11. The method of claim 1 further comprising:
processing the patient health data based on a predefined formula to generate a patient score;
processing the employee survey data based on a predefined formula to generate an employee score;
processing the patient score with the employee score to generate a schedule matrix; and
automatically populating the schedule matrix with at least one employee based on a predefined formula.

12. The method of claim 11 further comprising:
generating a schedule alert based on vacancies in the schedule matrix;
transmitting the schedule alert over a wireless communication channel to a wireless device associated with the at least one employee.

13. The method of claim 12 further comprising:
providing the at least one employee access to the employee portal in which the at least one employee may submit a request to fill a vacancy in the schedule matrix;
generating employee virtual currency based on a predefined formula.

14. The method of claim 11 wherein the patient health data may comprise initial prognosis data.

15. The method of claim 11 wherein the patient health data may comprise electronic medical records data.

16. The method of claim 1 further comprising:
accessing the administrator portal by the at least one administrator and awarding an amount of the virtual currency to the at least one employee virtual currency based on the employee survey data.

17. The method of claim 1 further comprising:
accessing the administrator portal by the at least one administrator and deleting the employee survey data based on an invalid survey score.

18. The method of claim 1 further comprising:
awarding employee virtual currency based on employee tenure.

19. The method of claim 1 further comprising:
awarding employee virtual currency based on employee referrals.

20. The method of claim 1 further comprising:
providing the at least one employee access to the employee portal in which the at least one employee may view an employee newsfeed.
